(19) United States
(12) Patent Application Publication
(10) Pub. No.: US 2009/0012563 A1
(43) Pub. Date: Jan. 8, 2009

(54) SPINAL FIXATION DEVICES AND METHODS

(75) Inventors: Neville Alleyne, La Jolla, CA (US); James R. Gerchow, Sturgis, MI (US)

Correspondence Address: KNOBBE MARTENS OLSON & BEAR LLP, 2040 MAIN STREET, FOURTEENTH FLOOR, IRVINE, CA, 92614, US

(73) Assignee: NAS Medical Technologies, Inc., Allen Park, MI (US)

(21) Appl. No.: 11/871,094

(22) Filed: Oct. 11, 2007

Related U.S. Application Data
(60) Provisional application No. 60/829,006, filed on Oct. 11, 2006.

(51) Int. Cl. A61B 17/70; A61B 17/08

(52) U.S. Cl. 606/246; 606/254; 606/151; 606/263; 606/261

(57) ABSTRACT

A rod, clamp and spinal fixation technique and method involving monoaxial or polyaxial screws for fusion in the cervical, thoracic, lumbar or sacral spine. The rod and clamp may be coupled to be pulled through the head of a fixation screw.

### CROSS REFERENCE TO RELATED APPLICATIONS

This application claims priority under 35 U.S.C. Section 119(e) to Provisional Application 60/829,006, filed on Oct. 11, 2006. The content of this provisional application is hereby incorporated by reference in its entirety.

### BACKGROUND OF THE INVENTION

1. Field of the Invention

This invention relates generally to orthopedic or neurosurgical devices and more specifically to a surgical devices and methods for vertebral fusion.

2. Description of the Related Art

Segmental pedicle screw fixation in the lumbar and thoracic spine has been well adopted over the last 30 years. Designs of different screws have allowed accommodation of a variety of sagittal and coronal angulations. The standard incisions have been midline incisions with the section of the soft tissues out to the tips of the transverse process. Occasionally, the surgeons will need to remove bone and ligament in order to gain access to the neural compromised region and/or remove disk material. When such decompressions are performed, iatrogenic instability is inevitable. As a result, fusion technology has evolved to place fixation screws and rods and bone material into particular areas of the spine in order to achieve successful fusion. The technology has evolved such that the incisions can be made smaller. Smaller incisions have created new challenges and difficulties for orthopedic and neurosurgeons. For example, it is difficult to visualize where to place the screws and to visualize where the passage of the rod occurs. Several devices over the years have evolved to allow the screws to be placed through biplane fluoroscopy. Fluoroscopy aligns the pedicle on both an AP and lateral view to facilitate passage of the K-wire and the cannulated screw to be placed into the pedicle quite reproducibly. However, the passage of the rod is difficult using minimally invasive surgery. Current technologies utilize unique dilators, cannulas or retractors in the wound in order to visualize where the rod is being placed. The “Sextant” system from Medtronic Sofamor Danek is an external apparatus that maintains the position of the two pedicle screws so that the rod can be passed through an arc of motion into the head of the pedicle screws. Some of these systems can be quite cumbersome and difficult to use.

### SUMMARY OF THE INVENTION

In one embodiment, a rod adapted for use in spinal fixation surgery comprises na elongate body having a first end and a second end and a coupler configured to accommodate an insertion apparatus at least at the first end.

In another embodiment, a clamp for passing a rod through a fixation screw during spinal fixation surgery comprises a body having a first end and a second end, an angled tip located at the first end configured to engage with an end portion of the rod, and a handle at the second end configured to enable a user to grip the clamp and produce an engagement between the clamp and the rod.

In another embodiment, a spinal fixation system comprises an elongate rod, a clamp with a tip that protrudes transversely from the body of the clamp, at least one fixation screw having a head with an opening, and a coupling that links an end of the rod and the tip of the clamp to enable passage of the rod through the opening of the fixation screw while attached to the clamp.

Methods of performing spinal fixation may comprise passing at least a portion of a clamp through an opening in a head of a fixation screw; engaging the clamp to a rod, pulling the rod through the opening of the fixation screw with the clamp, and disengaging the clamp from the rod.

In another embodiment, a spinal fixation system comprises a rod, a fixation screw having a head with an opening, a clamp, and means for coupling the clamp to an end portion of the rod through the opening such that pulling the clamp pulls the rod through the opening.

### BRIEF DESCRIPTION OF THE DRAWINGS

FIG. 1 is an example of a K-wire that is used to locate the pedicle under biplane fluoroscopy.

FIG. 2 is a pedicle screw cannulated at its desired diameter to allow a K-wire to pass freely.

FIGS. 3A-3D illustrates a clamp pulling a rod through two pedicle screws.

FIGS. 4A-4C illustrate one embodiment of a clamp that engages a recess in the end of the rod.

FIGS. 5A-5C illustrate another embodiment of a clamp that engages a recess in the end of the rod.

FIGS. 6A-6C illustrate another embodiment of a clamp that engages a recess in the end of the rod, wherein the clamp is formed from an elongated tube.

### DETAILED DESCRIPTION OF THE PREFERRED EMBODIMENTS

The present application is directed to spinal fixation devices and methods capable of being used in cervicothoracic, lumbar, sacral fusions. Instability can occur due to a fracture or spondylolisthesis, or internal disk derangement, or tumor, or ligamentous instability, or degenerative changes, or loss of bone from prior surgery such as a prior laminectomy with facetectomies, or iatrogenic instability from decompressive surgery for spinal stenosis, or removal of tumor, etc. When a spinal fusion procedure is performed, the orthopedic or neurosurgeon attempts to stabilize the posterior elements by placing pedicle screws and using a connecting rod or plate for segmental stability.

These constructs are then secured, and the corresponding facet joints and transverse prostheses are then decorticated and prepared for bone grafting material to be laid into the interstices of the intertransverse region and corresponding lateral aspect of the cassettes.

An embodiment of this invention allows the surgeon to place the screws percutaneously under fluoroscopic guidance or via open means. Once the pedicle is identified and prepared and checked to ascertain that there is no fracture of the walls anteriorly, superiorly, inferiorly or medially and laterally, then the corresponding pedicle screw is either inserted via a self tapping screw or a tap can be utilized and then the screw placed. Once the cannulated screw is placed or the non-cannulated screw is placed, the ability to pass a rod has been a difficult challenge for the surgeons. Attempts to pass the rod under biplane fluoroscopy can sometimes be difficult, time consuming, and lead to surgeon frustration. The devices described herein are designed to allow a clamp to fit through the pedicle screw whether it be monoaxial or polyaxial. This clamp can then be opened and the tip is capable of expanding to achieve a locking fit into the end of the rod. A device according to an embodiment of the invention operates with a rod in which the end of the rod has a female recess that allows the tip of the clamp to engage the rod. This initial docking of the clamp into the rod may allow the surgeon to hear an audible sound such as a “click.” In some embodiments, the surgeon can further expand the head of the clamp within the rod to achieve further stability and locking.

If a cannulated screw is utilized prior to passing the rod, the initial K-wire is removed. In a monoaxial or polyaxial open procedure or non-cannulated pedicle screw, there is no need to remove a K-wire. Those with skill in the art of orthopedics or neurosurgery would be able to place this clamp through any open screw and pull the rod or push the rod. They would then be able to achieve a stable construct with the pedicle screws and rods in satisfactory position. Once this is confirmed on x-ray or fluoroscopy or by direct visual means, the construct is then locked and tightened to its final inch pounds of torque for security.

While embodiments of the invention relate to spinal surgery, any fracture or stabilization in which a screw can be placed into a bone or bone-like structure, and a clamp is capable of pulling a rod or rod-like structure through it to achieve a degree of stability is within the scope of this invention.

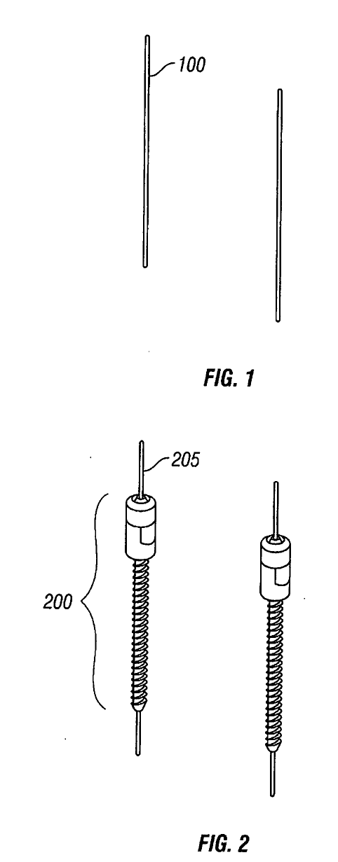

FIG. 1 illustrates an example of a K-wire 100 of any size that is capable of fitting into a cannulated monoaxial or polyaxial screw. The diameter of the K-wire 100 in FIG. 1 is 0.62 inches. The K-wire, however, could be of a plurality of sizes and diameters and is not limited by what is shown in the figure. The K-wire 100 may be a variety of lengths.

FIG. 2 illustrates an example of a cannulated pedicle screw 200 fitting over the K-wire 205 as it enters into the pedicle of the vertebra. As was discussed before, the ability to pass a K-wire into bone and have a screw placed over it does not limit us to the pedicle. We could also utilize this in a vertebral body, in a facet joint, in a spinous process or at the spinolaminar junction or in a long bone or flat bone of the human body. Once the desired location of the K-wire and pedicle screw is felt to be confirmed and satisfactory, the K-wire is then removed. One advantageous embodiment of the system comprises a pedicle screw, which can be monoaxial or polyaxial that has a cap nut already attached to the head of the pedicle screw with a set nut in the center, which can be tightened after the rod is passed through the pedicle screws. A pedicle fixation device of this type is described in U.S. patent application Ser. No. 10/411,075, published as U.S. Patent Publication 2004/0030337, the content of which is hereby incorporated by reference in its entirety.

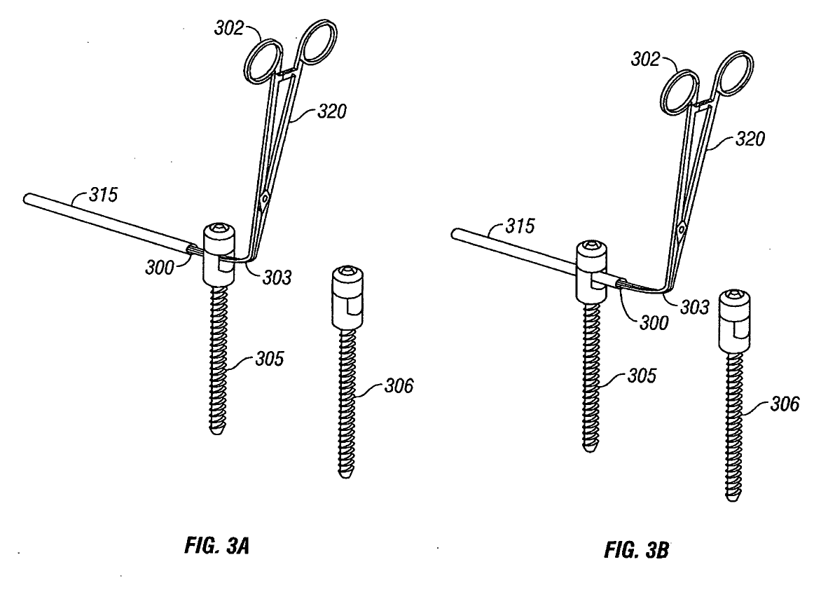

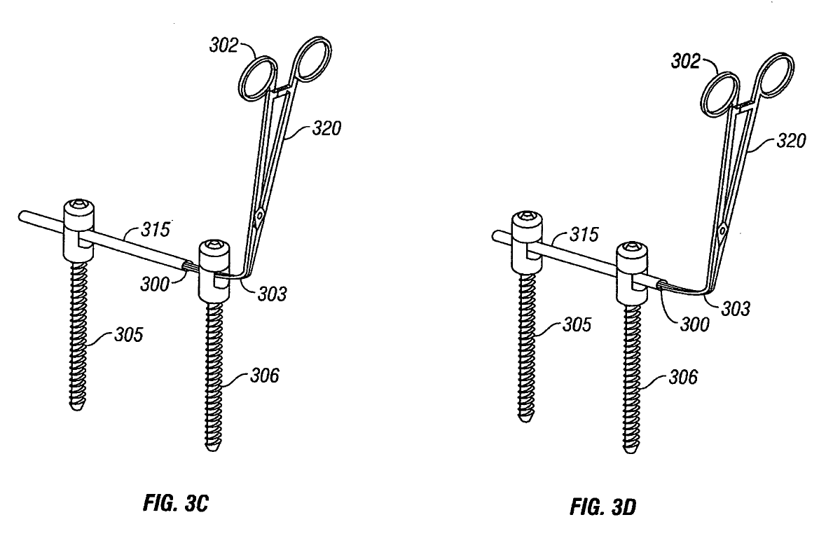

FIGS. 3A, 3B, 3C and 3D illustrate a clamp 303 according to an embodiment the invention that is angled at its tip 300 to pass through the opening in a monoaxial or polyaxial screw 305. As shown in this Figure, the clamp has the ability to pass through the opening of a pedicle screw 305 and is capable of opening or expanding at its tip 300 to grab the rod 315 and pull or push the rod 315 through the head of the pedicle screw 305.

In various embodiments of this invention, a plurality of tips to the clamp can be used. It is contemplated within the scope of this invention that the tip of the clamp may take on any number of forms or shapes, and those depicted in the figures are to be viewed as exemplary. The expandable tip of the clamp is designed to fit into a female opening on the end of the rod, which will then dock the clamp and rod. Docking may be associated with an audible “click”. In some embodiments, once the construct has docked, and the surgeon hears this audible clicking sound, the construct is provisionally locked. In some embodiments, in order to achieve better fixation, the clamp tip can then expand within the female end of the rod to lock the rod to the clamp to allow it to be pulled or pushed through the pedicle screw. Optionally, the tip of the clamp can also have a tapered end to grab onto a tapered end of a rod that may be flat or in a various plurality of configurations. This end of the rod, which can then be pulled through could also be disengaged from the main rod. This tip that is disengaged can then be removed after the rod is in its confirmed position.

In another embodiment of this invention, the device can be temporarily fixed to the end of the rod by a Morse taper that can then be disengaged at the end of the rod once it is in its confirmed position. In another embodiment, a wire or wire-like projection can extend from the end of the rod that allows a clamp to also pull the rod in a similar fashion through the head of the pedicle screw. In another embodiment, the rod is tapered to allow a flat end to be easily pulled through the end of a clamp that passes through the pedicle screw. In another embodiment, the rod has a telescopic end of decreasing sized diameters that is capable of being captured by a clamp that pulls it through the pedicle screw head. In another embodiment of this invention, the device is associated with a material that is capable of sticking to the end of the rod via a glue or glue-like substance to allow it to pull the rod through. Once this rod is in its confirmed position, the glue can then be heated or cauterized or cut with a scalpel or scissors to disengage the tip from the end of the rod. In another embodiment of this device, the clamp can be threaded into an end of the rod to pull the rod through the pedicle screw. Once the rod is in its confirmed position, the construct is tightened, and the clamp can then be unscrewed from the end of the rod.

In another embodiment of this device, the end of the rod can be fenestrated or have a recess or detent to allow a clamp to engage through or around the rod to pull the rod through the head of the pedicle screw to its desired position. Once this position is confirmed, the end of the rod can then be released by removing the end of the clamp from the detent.

In another embodiment of this invention, the head of the clamp can be engaged in such a way to lock into the end of the rod either through a female recess in the rod or a detent or fenestration in the rod and once this is engaged, the tip of the clamp can then be disengaged and left along with the rod in the wound.

The clamp may comprise a pair of arms 302 forming a handle that extend from the clamp 303 and attach to the main body of the clamp 303 as seen in FIGS. 3A-3D. The rod and clamps according to certain embodiments of the invention can be prepared from surgical steel, tungsten, titanium, tantalum, PMMA, PGA, PLA, PEEK (polyethyl ketone), or carbon or other suitable materials. The specification in no way limits the types of materials that may be used in the rods and clamps. The rod 315 can have an end that is modified in such a way to have fenestrations along the diameter or length of the rod 315 or may have detents along the end of the rod 315. These fenestrations or detents can be used also for anchoring points at adjacent levels if a longer construct is contemplated. As a result, the rod 315 can be pulled through to the cephalad or caudal adjacent pedicle, and a K-wire can then be passed through this fenestration in the rod to allow similar anchoring through the rod.

The actual dimensions of the clamp 303 will vary depending on a particular size of the monoaxial or polyaxial screw 310 being utilized. In one embodiment, the device is prepared in a size and shape to accommodate the standard 6.5 or 6.0 pedicle screw with a 0.065 cannulation. However, the screw could be used for the smaller diameter screw such as a 5.0 or 5.5 or 3.0 or 3.5 for the cervical and upper thoracic. This does not limit the possibility of larger diameters as well in the lower lumbar spine for 7.0, 7.5, 8.0, 8.5 or 9.0 in the sacrum. There are a plurality of different sizes for the clamp to come in to accommodate the openings of these various monoaxial and polyaxial screw heads.

The method and application of the device for any screw in which a rod or rod-like structure is being pulled through or pushed through openings in a pedicle screw is illustrated in the progression from FIGS. 3A to 3D. An expandable tip 300 of the clamp 303 is designed to fit into a female opening on the end of the rod 315, which will then dock the clamp 303 and rod 315. In FIG. 3A, the tip of the clamp 300 is positioned through an opening in pedicle screw 310. The tip 300 is engaged into the end of rod 315 using the expandable tip 300 and the female opening on the end of the rod 315. In FIG. 3B, the rod 315 is pulled through the opening in pedicle screw 305. The rod 315 is pulled until it reaches pedicle screw 306. In order to pull the rod 315 through pedicle screw 306, the tip 300 is collapsed and removed from the female opening on the end of rod 315. In FIG. 3C, the tip 300 is positioned through an opening in pedicle screw 306 and the tip 300 is engaged into the end of the rod 315. In FIG. 3D, we see the rod 315 being pulled through the opening in pedicle screw 306.

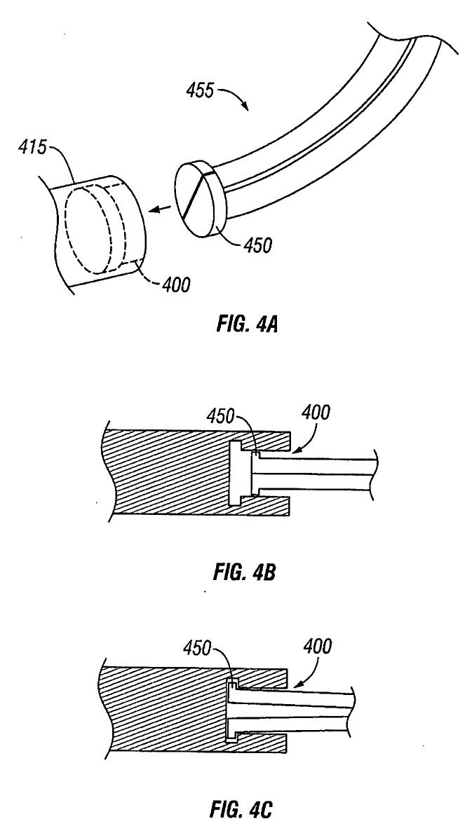

In the embodiment illustrated in FIGS. 3A to 3D, once the clamp 300 is released, the procedure can be done at 1, 2 or more levels. This allows the clamp 303 to be disengaged and placed into a cephalad or caudal vertebra to pull the rod 315 to the appropriate length for the motion segment or segments being fused. In this embodiment, the clamp 303 and rod 315 with the pedicle screws 305 and 306 is adapted to span at least 1 motion segment. It is contemplated that the construct can be prepared in a length and width to accommodate multiple motion segments being fused. In the embodiment illustrated in FIGS. 3A to 3D, the overall length of the rod 315 may be at least as long as 10 millimeters to accommodate 1 motion segment or may be as long as 500 millimeters to accommodate the entire cervical, thoracic, lumbar and sacral region. In some embodiments, the diameter of the rod 315 may vary from 2 millimeters all the way up to 8 millimeters in diameter. The recess at the end of the rod 315 may vary from 1 millimeter to 500 millimeters in depth to accompany a rod 315 that may span the entire length of the cervicothoracic and lumbar and sacral region. In this example, the rod 315 would have a design capable of being cannulated to its entire length. Customization of the rod 315 to the appropriate level of the fusion can be performed by using a tibial pin cutter or any such device that is well recognized by orthopedic or neurosurgeons. The design of the rod allows the end of the rod to be partially cannulated in its longitudinal axis or totally cannulated. However, in the preferred embodiments, as seen in FIGS. 4B and 4C, the rod 315 is only partially cannulated with its female adapted recess. It is contemplated that in one embodiment, the depth of the female recess to this rod may be approximately 1 millimeter to 15 millimeters.

This clamp 303 could have any of a variety of typical configurations for the handle 320 of the clamp 303 consistent with those recognized in the art of orthopedic or neurosurgery. Clamp lengths may vary from the handle and body. In certain embodiments, the clamp length may vary from 10 centimeters up to 300 centimeters from the handle to the body. The curved tip 300 may vary from 1 centimeter up to 40 centimeters. In certain embodiments, the length of the body would be 15 centimeters to 25 centimeters and for the curved tip the preferred length would be 5 centimeters to 30 centimeters.

FIG. 4A illustrates the end of a rod and a clamp according to an embodiment of the invention. The rod 415 has a female opening 400. The tip 450 of the clamp 455 is sized to enable it to pass through the opening in a pedicle screw and then into the recess 400 in the end of the rod as shown in FIG. 4B. The tip 450 is expandable as shown in FIG. 4C and allows the clamp to engage into the end of a rod according to an embodiment of this invention. The tip of the clamp may be capable of expanding up to two times its diameter when it is deployed in its opened and locked position versus when it is closed. It may be spring biased in the open position, such that it can be manually closed, inserted into the opening 400 in the rod, and then be allowed to snap into the expanded configuration of FIG. 4C when it reaches the proper depth into the shaped recess.

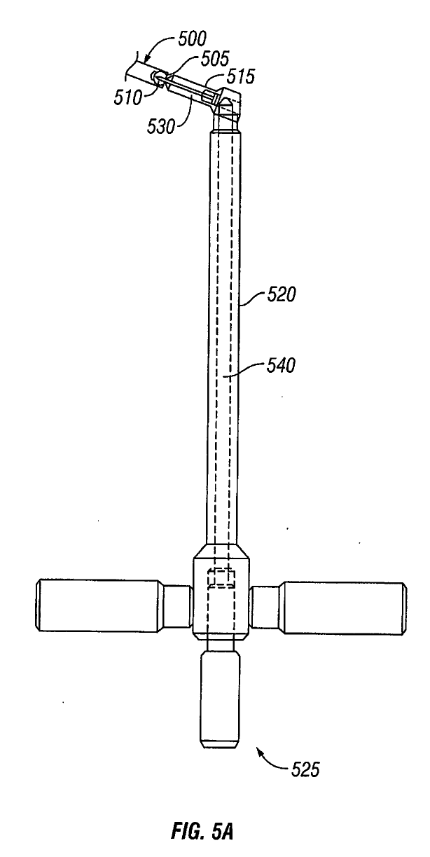

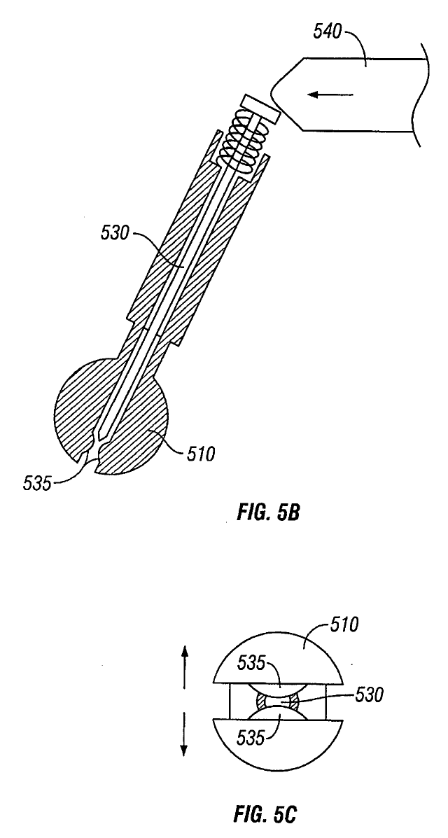

FIG. 5A illustrates a rod pulling clamp 520 according to another embodiment of the invention. The rod puller 520 has a clamp 515. The expandable tip 510 of the clamp 515 is engaged in the female opening 505 of rod 500. A handle 525 of the device may be used to expand or collapse the expandable tip 510 in order to allow the expandable tip 510 to engage or disengage from the female opening 505 of rod 500.

This is illustrated further in FIGS. 5B and 5C. FIG. 5B is a partial cutaway side view of the distal end of the rod puller clamp 520. FIG. 5C is an end view of the same distal end. The expandable tip 510 comprises a split ball that is expanded by forcing spike 530 to press against protrusions 535 on the inside of the split. The spike 530 is advanced against the protrusions by push rod 540 that can be threadably advanced by twisting the handle 525, or alternatively pushed. The tapered end of the push rod 540 pushes against the head of the spike 530, pushing the tip of the spike 530 between the protrusions 535 to expand the split tip. The end of the rod is provided with a corresponding recess that allows the tip in when in the collapsed position, and retains it inside the recess on a lip at the opening of the recess after the tip is expanded.

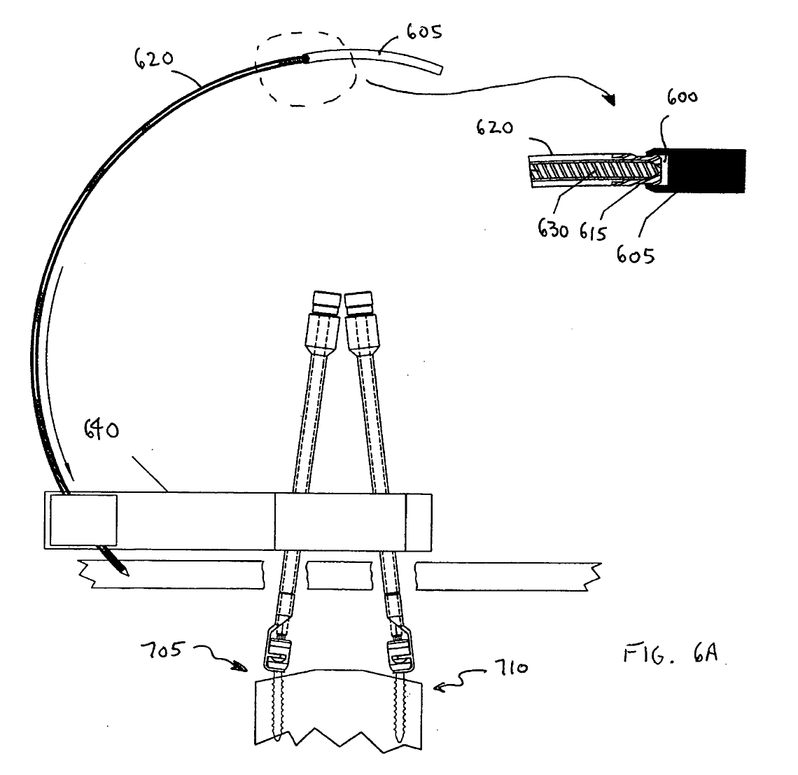

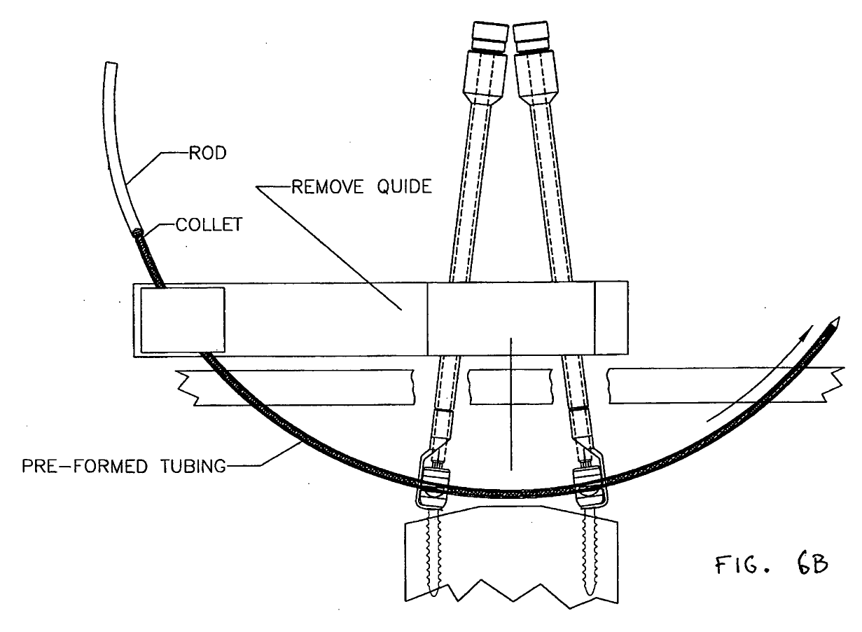

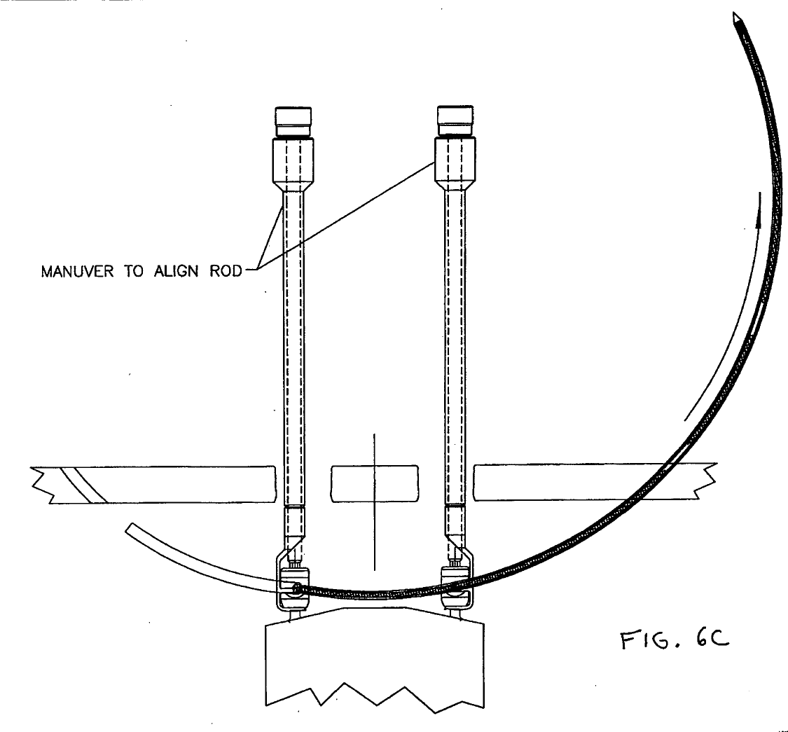

Another embodiment is illustrated in FIGS. 6A-6C. In this embodiment, a recess 600 in the end of the rod 605 is shaped to engage a mating collet 615 that is attached to the end of a length of tubing 620. The tubing can receive a flexible cable 630 that is threaded into the tubing 620 from the other side. When the end of the cable 630 is pushed against the inner surface of the collet 615, the collet 615 expands to lock behind the shoulder of the recess 600, thus securing the tubing 620 to the end of the rod 605.

As shown in FIGS. 6A-6C, the tubing 620 with the cable inserted and the collet attached to the rod is threaded through the heads of one or more fixation screws 705, 710. The tubing 620 is then pulled through the heads of the screws, thus pulling the rod 605 through the heads of the screws as well. During this procedure, the angles of the screw heads can be manipulated with additional tools to align the heads with the tubing and rod as they are pulled through. A guide can be used to place the entry point of the tubing relative to the screw positions. Various curvatures of tubing can be provided for different screw placements and depths.

Many embodiments of the invention allow the skin incision to remain small and for the surgeon to have an audible clicking to know that engagement and docking of the rod and clamp has occurred. Once that docking has occurred, the surgeon is then capable of deploying the tip of our clamp to expand within the rod to secure the lock fit. According to another embodiment of the invention, the rod will have both ends include the female recess to allow the clamp to pull from either caudal or cephalad position. In addition, traditional clamps known as rod holders may be used to assist in pushing or pulling the rod through the opening in the pedicle screw. In conjunction with the rod holders, there may be a head adjuster that will be capable of directing the pedicle screw head in the direction we would like for the rod. The head adjuster may also be aligned and marked such that whenever it is engaged, the head adjuster will be in alignment with the longitudinal axis of the rod. A particular pedicle screw may be utilized for the particular head adjuster. Such head adjusters already exist and are known to those who are performing these surgical techniques and procedures.

There are a variety of alternative adaptations and surgical procedures available to those of skill in the art for placement of these screws in the thoracic spine or placement of these screws in the lateral mass of the cervical spine posterior element. Pedicle screws can also be placed in the C2 pedicle or trans-articular screw fixation from C2 to C1 in the cervical spine. If any of these monoaxial or polyaxial screws are used in the cervical or thoracic spine as outlined, then the passage of the rod and docking of the rod with our clamp to engage and then finally lock the rod clamp unit for pushing or pulling through these pedicle screws is what is being described.

The above described technique is not only for minimally invasive surgeons but can be used by surgeons who are doing standard open techniques, surgeons who want to work through a smaller incision, and also surgeons who want to do strict minimally invasive surgery. While particular embodiments of the invention and method have been described in detail, it will be apparent to those skilled in the art that these embodiments are exemplary rather than limiting.

What is claimed is:

1. A rod adapted for use in spinal fixation surgery, the rod comprising:
a elongate body having a first end and a second end; and
a coupler configured to accommodate an insertion apparatus at least at the first end.

2. The rod of claim 1, comprising a recess configured to accommodate an insertion apparatus.

3. The rod of claim 2, wherein the recess comprises an end portion and an interior portion, and the recess is larger at the interior portion than the end portion.

4. The rod of claim 3, wherein the recess is approximately spherical.

5. The rod of claim 1, wherein the end of the rod comprises a Morse taper.

6. The rod of claim 1, comprising a wire or wire-like projection extending from an end of the rod.

7. The rod of claim 1, wherein the end of the rod is configured with a telescopic end of decreasing size diameters.

8. A clamp for passing a rod through a fixation screw during spinal fixation surgery, the clamp comprising:
a body having a first end and a second end;
a tip located at said first end configured to engage with an end portion of the rod; and
a handle at the second end configured to enable a user to grip the clamp and produce an engagement between the clamp and the rod.

9. The clamp of claim 8, wherein the handle is configured to allow the user to tighten or loosen the engagement of the angled tip with the rod.

10. The clamp of claim 8, wherein the tip of the clamp is expandable.

11. The clamp of claim 8, wherein the clamp is curved or bent.

12. A spinal fixation system, the system comprising:
an elongate rod;
a clamp with a tip;
at least one fixation screw having a head with an opening; and
a coupling that links an end of the rod and the tip of the clamp to enable passage of the rod through the opening of the fixation screw while attached to the clamp.

13. The system of claim 12, wherein the coupling comprises a male end on the tip of the clamp configured to engage a female opening on the end of the rod.

14. The system of claim 13, wherein the coupling comprises a collet.

15. The system of claim 12, wherein the clamp comprises a tube with a collet affixed to the end thereof.

16. The system of claim 12, wherein the coupling is configured to produce tactile feedback when the end of the rod and the tip of the clamp are linked.

17. A method for performing spinal fixation, the method comprising:
passing at least a portion of a clamp through an opening in a head of a fixation screw;
engaging the clamp to a rod;
pulling the rod through the opening of the fixation screw with the clamp; and
disengaging the clamp from the rod.

18. The method of claim 17, comprising performing a stab incision over or adjacent to the head of the fixation screw to enable passage of the tip of the clamp through the opening in the head of the fixation screw.

19. The method of claim 17, further comprising securing the rod onto the head of the fixation screw when the rod is located in its desired position.

20. The method of claim 17, additionally comprising:
passing at least a portion of the clamp through an opening in a head of a second fixation screw;
engaging the clamp to the rod;
pulling the rod through the opening of the second fixation screw with the clamp; and
disengaging the clamp from the rod.

21. The method of claim 17, wherein the engaging is performed prior to the passing.

22. A spinal fixation system comprising:
a rod;
a fixation screw having a head with an opening;
a clamp; and
means for maintaining the clamp fixed to an end portion of the rod while the clamp extends through the opening, whereby forcing clamp through the opening forces the rod through the opening.

23. The spinal fixation system on claim 22, wherein the rod comprises a recess in an end portion thereof.

24. The spinal fixation system of claim 23, wherein the clamp comprises an expandable tip configured to engage the recess.

25. The spinal fixation system of claim 17, wherein the clamp comprises a tube with a collet affixed to an end thereof.
